(19) United States
(12) Patent Application Publication
(10) Pub. No.: US 2018/0104283 A1
(43) Pub. Date: Apr. 19, 2018

(54) COSMETIC SKIN TREATMENT METHOD

(71) Applicant: Gregory Gary Caputy, Honolulu, HI (US)

(72) Inventor: Gregory Gary Caputy, Honolulu, HI (US)

(21) Appl. No.: 15/292,335

(22) Filed: Oct. 13, 2016

(51) Int. Cl. A61K 35/74

(52) U.S. Cl. CPC A61K 35/74

(57) ABSTRACT

A method for treating and preventing topical skin conditions by establishing a robust normal bacterial and/or microbiota population on the treated skin is provided.

### BACKGROUND OF THE INVENTION

The present invention relates to cosmetic treatment methods and, more particularly, to a method of treating skin-related medical conditions by establishing a diverse normal bacterial population on the skin, thereby preventing the colonization of “bad” bacteria that is at the root of the skin-related medical conditions being treated.

There are multiple superficial, skin-related medical conditions, such as acne, dyshidrosis, cradle cap, foot odor, etc., which in part result from disproportional growth of “bad” bacteria along the affected skin. Importantly, though, is the presence of not just bad bacteria but also normal bacterial flora or microbiota, which includes a population of “good” bacteria the skin and body need, benefit from, form a commensal relationship with, or at worst are innocuous therewith.

The current treatments of such skin-related medical conditions, however, do not address the abovementioned root cause. In some instances, only the symptoms are addressed. Other approaches disadvantageously address the disproportional growth by indiscriminatingly killing the overall population of bacteria, thereby reducing the good bacteria, opening the door for the bad bacteria to reclaim its stake as well as expand its original territory by growing along skin once inhabited by the good bacteria. This happens because skin bacteria are never permanently eradicated as skin cannot be permanently sterilized; rather, healthy skin promotes bacteria growth. And in some instances the first bacteria back are usually the bad ones-like weeds in a field.

Examples of such current treatments including the use of antibiotic and antibacterial skin cleansers have been offered as a treatment. An additional disadvantage with antibiotic use, besides the world-wide problems it is causing, is its overuse leads to antibiotic-resistant organisms, rendering such treatment ineffective.

Moreover, current acne treatments try to kill bacteria typically by using peroxide. Underarm sprays or foot sprays mask the odor or decrease wetness but do nothing to establish a normal bacterial flora, which neither produce substances which cause odor nor produce free fatty acids, irritating the skin and causing acne and other irritations.

As can be seen, there is a need for a method of treating skin-related medical conditions by establishing a robust normal bacterial population on the skin, thereby preventing the colonization of “bad” bacteria that is at the root of the skin-related medical conditions being treated. Whereby, the robust normal bacterial population out-competes the bacterial species which cause the noted conditions, whereby the normal bacterial flora on the skin prevents or stymies the bad bacteria colonization in a natural, healthy way.

### SUMMARY OF THE INVENTION

In one aspect of the present invention, a method for treating a plurality of topical skin conditions includes applying a first microbiota on a dermal environment affected by at least one of the plurality of topical skin conditions, whereby the first microbiota prevents the colonization of the dermal environment by an initial bacteria causing the at least one of the plurality of topical skin conditions.

In another aspect of the present invention, the method for treating a plurality of topical skin conditions includes freeze-drying commensal bacteria; solubilizing the commensal bacteria with an aqueous medium, activating the commensal bacteria; applying commensal bacteria on a dermal environment affected by at least one of the plurality of topical skin conditions, wherein the activated commensal bacteria is adapted to establish a topical microenvironment of reduced oxygen tension along the dermal environment; and applying a first microbiota and/or further microbiota on said topical microenvironment, wherein the first microbiota and/or further microbiota are adapted to prevent the colonization of the dermal environment by the initial bacteria causing the at least one of the plurality of topical skin conditions.

In yet another aspect of the present invention, the first microbiota and/or further microbiota are applied over several days, wherein the initial bacteria includes Propionobacterium, wherein the first microbiota and/or further microbiota includes at least one of the following: bacteria, fungi or viruses, and/or wherein the dermal environment is disposed along at least one of the following: portions of a gut, an oral cavity portion, or a vaginal portion.

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method for treating and preventing topical skin conditions by establishing a robust normal bacterial and/or microbiota population on the treated skin.

The present invention may include a method of treating skin-related medical conditions by establishing a robust normal bacterial population on the treated skin. The present invention includes providing and cultivating various strains of non-harmful skin bacteria which are known to be commensal, present on the majority of people's skin and known to be harmless and abundant in nature. The various strains of non-harmful skin bacteria may be activated from a freeze-dried state by mixing with an aqueous gel, the resulting mixture is then applied to a treatment area/skin environment. Various other bacteria are sequentially added after the skin environment is so conditioned, ready to support a complex, mixed flora population, which would not have lived on their own had they been applied first. An analogy would be akin to growing a rain forest; not all the species can be added at once as some need to be established prior to adding new species in order to condition the environment for the eventual survival of the latter.

As the dermal environment is conditioned, there are complex microenvironments that are changed for supporting bacterial growth. By way of example, hair bearing skin of the underarms has very different microbial populations than the relatively hairless skin of the nose and that is different from the skin behind the ears. However, by applying mixed cultures of commensals and gradually conditioning the environments, the optimal conditions for growth, as varied as it is, is possible and can predictably be reached. Returning to the ecological analogy, a key characteristic in a healthy ecology is a robust diversity of species and organisms, be it a rain or deciduous forest. Through supporting different species, both types of forest are equally appropriate for their environment and thus more resistance to certain adverse growth. In contrast, a field of grass, although somewhat protective against the growth of weeds is much less so, much more sensitive to small changes in the environment and is much more readily completely eradicated than a complex mixture of organisms. It is a common principle of ecology.

The richness of the normal flora of skin is just beginning to be elucidated with the Human Microbiome Project (being run by the NIH for a the last several years). They are finding the normal flora very complex, wherein there is almost a “fingerprint” of bacteria for each person. What has not yet been discovered or appreciated, though, is that there are underlying bacteria common to establishing a healthy, complex human flora growth, a growth which can vary from microenvironment to microenvironment on the skin.

The present invention proposes a route to establish a complex flora on the skin by using aerobic organisms to establish a topical microenvironment of reduced oxygen tension. The aerobic organisms use oxygen in their metabolic pathways which lowers the adjacent oxygen tension thus making a facultative and, even anaerobic environment at the base of hair follicles and pores, whereby facultative and anaerobic organisms can be applied to and survive. Namely, freeze-drying common strains of the correct organisms in the correct sequence of application with the aqueous gel for rehydration and keeping the organisms alive. The product of the freeze-dried bacteria to add to the aqueous gel for activation would be highly varied.

A method of using the present invention may include the following. A young woman, for example, beginning to get a few pimples at puberty would begin with step one of the series by applying live bacteria to her skin nightly. The packet of freeze-dried bacteria is added to gel and mixed for a few moments. The gel with activated, live bacteria are now spread over the skin and left in place-as a mask for several minutes. The bacterial species added to the gel would then be changed, slowly, over months so as to provide the correct bacteria at the correct time for a complex microbial flora to be established. This would out-compete the Propionobacterium acnes which is the “bad”, anaerobic species causing her early acne. After a few months, a normal flora is established and she is protected from acne. The process would be similar for someone with smelly feet, axillae, or the like. Additionally, the true bacterial species of the gut, once properly elucidated, could be used in a similar manner to establish normal gut flora as babies grow and progress through eating new foods. Many food “allergies” and colic could be avoided or treated in this manner.

In one aspect of the present invention, freeze-dried bacteria are solubilized with an aqueous medium, with various steps and stages for various times in order to reestablish a robust normal skin flora.

In another aspect of the present invention, babies have normal vaginal flora, similarly rehydrated, applied over several days to establish a normal flora of the skin. This is particularly important in newborns born by caesarean section. It being understood that in the instance of vaginal flora or gut flora, that the initial bacterium may not be S. epidermidis-which is typically for the skin instance only. Gut flora would likely be some form of lactobacillus (as in probiotic yogurts), similarly with vaginal flora. Oral flora would begin with streptococci and lactobacilli in those persons with erupted teeth and, perhaps, periodontitis and rehydration would occur with some type of aqueous mouthwash. Also, the present invention does not preclude the need to first, as much as possible, reduce the bacterial concentration on the surface prior to beginning the application of the live bacteria. This could be something as simple as using a deodorant soap or topical antibacterial cleanser for a few days in the case of the skin or a mouthwash in the case of the oral cavity.

It should be understood, of course, that the foregoing relates to exemplary embodiments of the invention and that modifications may be made without departing from the spirit and scope of the invention as set forth in the following claims.

1. A method for treating a plurality of topical skin conditions, by first establishing a topical microenvironment of reduced oxygen tension, comprising the steps of:
applying a first microbiota on a dermal environment affected by at least one of the plurality of topical skin conditions, whereby the first microbiota is non-yogurt based, and is only microbiota commensal with and prevents the colonization of the dermal environment by an initial bacteria causing the at least one of the plurality of topical skin conditions.

2. The method of claim 1, wherein the first microbiota comprises aerobic organisms adapted to establish a topical microenvironment of reduced oxygen tension along the dermal environment.

3. (canceled)

4. The method of claim 1, wherein the first microbiota includes freeze-dried bacteria solubilized with an aqueous medium.

5. The method of claim 4, wherein the aqueous medium activates the freeze-dried bacteria solubilized.

6. The method of claim 1, further comprising the step of:
applying a second microbiota on said dermal environment, whereby the second microbiota prevents the colonization of the dermal environment by the initial bacteria causing the at least one of the plurality of topical skin conditions.

7. The method of claim 6, further comprising the step of:
sequentially applying a third microbiota and/or further microbiota on said dermal environment, wherein the third microbiota and/or further microbiota prevents the colonization of the dermal environment by the initial bacteria causing the at least one of the plurality of topical skin conditions, and wherein the third microbiota and/or further microbiota are applied over several days.

8. The method of claim 1, wherein the initial bacteria includes Propionobacterium.

9. The method of claim 1, wherein the first, a second, a third and/or further microbiota includes at least one of the following: bacteria, fungi or viruses.

10. The method of claim 1, wherein the dermal environment is disposed along at least one of the following: portions of an oral cavity portion or a vaginal portion.

11. A method for treating a plurality of topical skin conditions, comprising the steps of:
freeze-drying commensal bacteria;
solubilizing the commensal bacteria with an aqueous medium, activating the commensal bacteria;
applying commensal bacteria on a dermal environment affected by at least one of the plurality of topical skin conditions, wherein the activated commensal bacteria is non-yogurt based and adapted to establish a topical microenvironment of reduced oxygen tension along the dermal environment; and
applying a first microbiota and/or further microbiota on said topical microenvironment, wherein the first microbiota and/or further microbiota are adapted to prevent the colonization of the dermal environment by the initial bacteria causing the at least one of the plurality of topical skin conditions.

12. The method of claim 11, wherein the first microbiota and/or further microbiota are applied over several days.

13. The method of claim 11, wherein the initial bacteria includes Propionobacterium.

14. The method of claim 11, wherein the first microbiota and/or further microbiota includes at least one of the following: bacteria, fungi or viruses.

15. The method of claim 11, wherein the dermal environment is disposed along at least one of the following: portions of an oral cavity portion or a vaginal portion.
